(19) United States
(12) Patent Application Publication
(10) Pub. No.: US 2019/0254944 A1
(43) Pub. Date: Aug. 22, 2019

(54) Skin Care Compositions

(71) Applicant: Jamie Nicole Baca, Santa Monica, CA (US)

(72) Inventor: Jamie Nicole Baca, Santa Monica, CA (US)

(21) Appl. No.: 16/402,104

(22) Filed: May 2, 2019

Related U.S. Application Data
(62) Division of application No. 15/934,470, filed on Mar. 23, 2018.
(60) Provisional application No. 62/476,677, filed on Mar. 24, 2017.

(51) Int. Cl. A61K 8/365; A61K 8/37; A61Q 19/08; A61Q 19/02; A61K 8/42; A61K 8/73; A61K 8/368; A61K 8/67; A61K 8/34; A61K 8/362

(52) U.S. Cl. CPC A61K 8/365; A61K 8/375; A61Q 19/08; A61Q 19/02; A61K 8/42; A61K 2800/42; A61K 8/368; A61K 8/671; A61K 8/345; A61K 8/347; A61K 8/362; A61K 8/73

(57) ABSTRACT

A skin care composition comprising less than 0.05% lactic acid, about 5% to about 10% sodium lactate, about 1% to about 5% niacinamide, less than about 0.1% biosaccharide gum-2, about 0.1% to about 1% bakuchiol, about 0.1% to about 1% azelaic acid, less than about 0.1% retinol, and less than about 0.1% lecithin.

### CROSS-REFERENCE TO RELATED APPLICATIONS

The present application is a divisional of U.S. patent application Ser. No. 15/934,470 entitled “Skin Care Compositions,” filed Mar. 23, 2018, and the present application claims the benefit of U.S. Provisional Patent Application No. 62/476,677 entitled “Skin Care Composition,” filed Mar. 24, 2017, the contents of which are incorporated in this disclosure by reference in their entirety.

### BACKGROUND

Billions of dollars are spent annually on anti-aging products. As a large percent of the population ages and is confronted with a youth oriented culture, the anti-aging market has seen a compounded annual growth rate of 7.8% between 2013 and 2019 and is projected to be worth USD 191.7 billion by 2019. See, e.g., “Anti-aging Market—Global Industry Analysis, Size, Share, Growth, Trends and Forecast 2013-2019”. Anti-aging skin care compositions are a large part of this market. Anti-aging skin care sales in the United States generates about 2.1 billion U.S. dollars annually in retail sales. People are constantly looking for products to improve the youthful and overall appearance of their skin and reduce the visible signs of aging, including reducing the appearance of wrinkles, age spots, and rosacea, and at the same time have a healthy youthful glow.

A variety of products are on the market to address these issues, including retinol based products (to reduce the appearance of wrinkles); hydrating lotions and creams, and bleaching or lightening creams (such as hydroquinone, or enzyme formulations). However, these products suffer from one or more disadvantages including irritation from harsh ingredients, dryness, and/or not being not addressing multiple signs of aging, such as the both appearance of wrinkles and age spots.

### SUMMARY

In a first embodiment, the invention is directed to a skin care composition comprising less than 0.1% lactic acid, about 5% to about 10% sodium lactate, about 1% to about 5% niacinamide, less than about 0.1% biosaccharide gum-2, about 0.1% to about 1% bakuchiol, about 0.1% to about 1% azelaic acid, less than about 0.1% retinol, and less than about 0.1% lecithin.

Optionally, the skin care composition comprises one or more additional ingredients selected from the group consisting of water, polyacrylate-13, cyclopentasiloxane, isoamyl laurate, magnesium aluminum silicate, panthenol, glycerin, phenyl trimethicone, dicaprylyl carbonate, polyisobutene, dimethicone, polysorbate 80, hydroxyethylcellulose, polysorbate 20, pentylene glycol, phenoxyethanol, laureth-4, ethylhexylglycerin, tetrasodium glutamate diacetate, and potassium hydroxide.

Optionally, the skin care composition further comprises more than about 50/o water, about 1% to about 5% polyacrylate-13, about 0.1% to about 1% polyisobutene, about 0.1% to about 1% polysorbate 20, about 1% to about 5% cyclopentasiloxane, about 0.1% to about 1% dimethicone, about 1% to about 5% magnesium aluminum silicate, about 1% to about 5% isoamyl laurate, about 1% to about 5% panthenol, about 1% to about 5% glycerin, about 1% to about 5% pentylene glycol, about 0.1% to about 1% phenoxyethanol, about 0.1% to about 1% phenyl trimethicone, about 0.1% to about 1% dicaprylyl carbonate, about 0.1% to about 1% potassium hydroxide, about 0.1% to about 1% polysorbate 80, about 0.1% to about 1% hydroxyethylcellulose, about 0.1% to about 1% laureth-4, about 0.1% to about 1% ethylhexylglycerin, and less than about 0.1% tetrasodium glutamate diacetate.

In a second embodiment, skin care composition comprises lactic acid, niacinamide, biosaccharide gum-2, bakuchiol, azelaic acid, and retinol.

Optionally, the lactic acid comprises less than 0.1% of the skin care composition, the niacinamide comprises from about 1% to about 5° % of the skin care composition, the biosaccharide gum-2 comprises less than about 0.1% of the skin care composition, the bakuchiol comprises from about 0.1% to about 1% of the skin care composition, the azelaic acid comprises from about 0.1% to about 1% of the skin care composition, and the retinol comprises less than 0.1% of the skin care composition.

Optionally, the skin care composition comprises one or more additional ingredients selected from the group consisting of water, polyacrylate-13, cyclopentasiloxane, isoamyl laurate, magnesium aluminum silicate, panthenol, glycerin, phenyl trimethicone, dicaprylyl carbonate, polyisobutene, dimethicone, polysorbate 80, hydroxyethylcellulose, polysorbate 20, pentylene glycol, phenoxyethanol, laureth-4, ethylhexylglycerin, tetrasodium glutamate diacetate, potassium hydroxide, sodium lactate and lecithin.

Optionally, the skin care composition further comprises more than about 50% water, about 1% to about 5% polyacrylate-13, about 0.1% to about 1% polyisobutene, about 0.1% to about 1% polysorbate 20, about 1% to about 5% cyclopentasiloxane, about 0.1% to about 1% dimethicone, about 1% to about 5% magnesium aluminum silicate, about 1% to about 5% isoamyl laurate, about 1% to about 5% panthenol, about 1% to about 5% glycerin, about 1% to about 5% pentylene glycol, about 0.1% to about 1% phenoxyethanol, about 0.1% to about 1% phenyl trimethicone, about 0.1% to about 1% dicaprylyl carbonate, about 0.1% to about 1% potassium hydroxide, about 0.1% to about 1% polysorbate 80, about 0.1% to about 1% hydroxyethylcellulose, about 0.1% to about 1% laureth-4, about 0.1% to about 1% ethylhexylglycerin, and less than about 0.1% tetrasodium glutamate diacetate, about 5% to about 10% sodium lactate and less than about 0.1% lecithin.

### DESCRIPTION

As used herein, the following terms and variations thereof have the meanings given below, unless a different meaning is clearly intended by the contest in which such term is used.

The terms “a,” “an,” and “the” and similar referents used herein are to be construed to cover both the singular and the plural unless their usage in context indicates otherwise.

As used in this disclosure, the term “comprise” and variations of the term, such as “comprising” and “comprises,” are not intended to exclude other additives, components, integers ingredients or steps.

According to the present invention, a skin care composition is provided which addresses multiple visible signs of aging and improves the appearance of the skin. In some embodiments, the skin care composition acts as a primer and leaves the skin suitable for make-up application after application of the skin care composition.

Anti-aging and brightening skin care products are known. However, known compositions do not have the combination of multiple active ingredients, such as brighteners, anti-oxidants, wrinkle reducers and hydrators, which the present invention combines into an all-in-one product. Compositions with multiple active ingredients, such as the present invention described herein, have generally not been formulated (or are not commercially available) due to the cost of the active ingredients in the product, and the difficulty in the formulation. The difficulty in combining multiple ingredients lies in combining these ingredients in a formulation which is potent (meaning, has a high percentage of active ingredients), but still has an appropriate texture, spreadability and does not decompose over time. The skin care compositions according to the present invention have been formulated with multiple active ingredients in a potent formulation which, when tested over time, shows stability at 2, 4, 8 and 12 weeks. In addition, the skin care composition of the present invention has been consumer tested to show superior results in the appearance of the skin over time.

According to one embodiment, the skin care composition comprises a combination of an alpha-hydroxy acid (AHA) and/or salts and esters thereof, a stabilized retinol and a retinol-like compound, one or more saturated dicarboxylic acids, and Vitamin B3. The composition may additionally contain one or more polysaccharides or biosaccharides and other ingredients including but not limited to moisturizing active ingredients, hydrators, penetration enhancers, emulsifiers, thickeners, feel modifiers, sensory enhancers, and stability enhancers to enhance the composition performance, stabilization, texture, viscosity and spreadability.

The skin care composition comprises one or more alpha hydroxy acids (AHAs) which act as wrinkle-smoothers and also have been shown to help clear blemishes, hydrate the skin and lighten pigment spots by loosening one to several layers of dead cells clinging to the skin's surface. Preferred AHAs include azelaic acid, glycolic acid, lactic acid, tartaric acid, malic acid, citric acid, gluconic acid and derivatives thereof. More preferably, the AHA is present in the composition from 0.05% to 30%.

Most preferably, the AHA is azelaic acid, which has been found to be less reactive on the skin that other AHAs and provides mild exfoliation without irritation. Azelaic acid (1,7-hepanedicarboxylic acid) is a naturally occurring dicarboxylic acid. It occurs amongst others in cereals like wheat, barley and rye. Extracts of these cereals have been used since 3000 years in Ayurveda products to treat hyperpigmentation (skin lightening). Azelaic acid also exhibits bactericidal properties, and was shown to be very effective for the treatment of acne vulgaris. Azelaic acid obtained from natural resources is not commercially available; the extraction process is very difficult and expensive. For this invention, azelaic acid is produced by ozonisation of oleic acid. The oxidation of oleic acid results in the formation of azelaic acid and nonanoic acid. The oleic acid required is obtained from hybrid sunflower oil and milk thistle oil. This choice of starting material enables production of azelaic acid with a purity grade of more than 99%, whereas regular azelaic purity grades are specified at 85-90%. Azelaic acid is an odorless, white and highly crystalline solid with a melting point of 110° C. It is soluble in glycerol and glycols, but poorly soluble in water (2.1 g/l). The solubility of azelaic acid is pH dependent; the mono- and disodium salts are significantly better. Also micro-emulsions based on sorbitan esters using glycerol and lauryl alcohol as co-surfactants enable to dissolve azelaic acid. The azelaic acid utilized in the present invention is unique because it is a hybrid of oleic acid and milk thistle. Azelaic acid is an organic compound with the formula HOOC(CH2)7COOH. This saturated 1,7-heptanedicarboxylic acid exists as a free flowing micronized white to slight yellow crystal powder and is 100% vegetable origin. Azelaic acid is used to treat mild to moderate acne, both comedonal acne and inflammatory acne. It belongs to a class of medication called dicarboxylic acids. It works by killing acne bacteria that infect skin pores. It also decreases the production of keratin, which is a natural substance that promotes the growth of acne bacteria. Azelaic acid is also used as a topical gel treatment for rosacea, due to its ability to reduce inflammation. It clears the bumps and swelling caused by rosacea. The mechanism of action is thought to be through the inhibition of hyperactive protease activity that converts cathelicidin into the antimicrobial skin peptide LL-37. Azelaic acid has been used for treatment of skin pigmentation including melasma and postinflammatory hyperpigmentation, particularly in those with darker skin types. It can be used as an alternative to hydroquinone. As a tyrosinase inhibitor, azelaic acid reduces synthesis of melanin.

The skin care composition also comprises one or more salts of AHAs which act a humectant to bind and hold water in the stratum corneum (the upper layer of the skin) and is a natural moisturizing factor. The higher the water content of the stratum corneum, the more plasticized it is, and the more soft and supple the skin becomes. Salts of AHAs include glycolate, lactate, tartarate, malate, citrate and gluconate. Preferably, the skin care composition comprises an AHA which is effective, and due to consumer perceptions of safety, is a natural product from a natural source. More preferably, the skin care composition comprises a salt based on natural L-Lactic acid. A suitable L-Lactic acid formulation for use in the composition is available from Corbion Purac, which is produced by fermentation of biobased carbohydrates.

The skin care composition also comprises one or more Retin A (tretinoin), retinols or retinol-like compounds, and preferably, a combination of a retinol and a retinol-like compound. Compounds such as Retin-A® and tretinoin and other retinols, such as isotretinoin, etretinate, and acitretin, have been shown to increase skin cell turnover and stimulate collagen synthesis. However, these compounds also have issues with skin irritation, skin sensitization, and teratogenicity, as well as break down of the compounds when stored over time in the formulated compositions. Additionally, these compounds can have a photochemical and hydrolytic instability, meaning their use is primarily restricted to nighttime. Preferably, the skin care composition includes a first retinol, preferably in a stabilized liposomal form, to promote skin cell turnover and collagen production, and a second retinol-like compound, which serves as a broad spectrum anti-oxidant and anti-inflammatory. The combination of the two retinols has been found to enhance the reduction in the appearance of wrinkles and age spots, while minimizing irritation. More preferably, the skin care composition includes a first retinol, a stabilized Vitamin A liposome, such as VitAine, a Vitamin A liposome containing a 0.5% stabilized retinol, available from Barnet products corporation. Preferably, the first retinol is a 0.5% liposomal stabilized retinol present in the composition in an amount less than 0.1%. In the event the first retinol is a (tretinoin), preferably the tretinoin is present in the composition in an amount from about 0.025% to about 0.1%, more preferably from about 0.05% to about 0.01%. The second retinol-like compound, such as Sytenol A, (i.e, bakuchiol), is available from Sythen Ltd, and is preferably present in the composition from about 0.01% to about 3%, and more preferably, from about 0.1% to about 1%, most preferably, about 0.5%. Bakuchiol is effectively quenches superoxide-, hydroxyl-, peroxy-, peroxynitrile radicals and singlet oxygen non-radical in addition to inhibiting lipid peroxidation. Bakuchiol is also very effective in increasing mitochondrial biogenesis and protecting mitochondrial functions against oxidative stress. Bakuchiol can also upregulate the endogenous antioxidant defense system. Bakuchiol is devoid of safety issues, compatible with a wide variety of cosmetic ingredients and is devoid of instability problems.

The skin care composition also comprises one or more saturated dicarboxylic acids of the formula HOOC—(CH2) n-COOH, and derivatives thereof, including adipic acid, pimelic acid, suberic acid, azelaic acid, sebacic acid, dodecanedoic acid, and derivatives thereof. Preferably, the skin care composition comprises azelaic acid obtained from a natural source, and is a grade of more than 99% pure, and is present in the composition from about 0.01% to about 30%, and more preferably, from about 0.1% to about 5%, most preferably about 0.1%. A suitable azelaic acid formulation is available from Azelaic Products.

The skin care composition also comprises one or ingredients that reduce the transepidermal water loss, such as Vitamin B3 (niacinamide). It has been found that skin moisturization, skin softness, suppleness and skin hydration are related to the barrier properties of the horny layer. It is known that several lipids such as fatty acids and ceramides are critical for the structural and functional integrity of the stratum corneum. The skin barrier function can be assessed by transepidermal water loss (TEWL) measurements. It has been found that niacinamide reduces TEWL by 24% in 4 weeks, and at the same time boosts free fatty acids and ceramides in the stratum corneum. In addition, niacinamide stimulates the brightening or clearing of the skin (reducing pigmentation). Preferably, the niacinamide is present in the composition from about 0.5% to about 5%, more preferably, from about 2% to about 4%, and most preferably about 40%. A suitable niacinamide formulation is available from DSM Nutritional products.

Optionally, instead of niacinamide, hydroquinone can be used. Hydroquinone (benzene-1,4-diol) brightens patches of darkened skin, freckles and age spots by decreasing the formation of melanin in the skin. Preferably, hydroquinone is present in the composition from about 0.5% to about 6%, more preferably from about 1% to about 4%, and most preferably about 40%.

The skin care composition may further comprise one or more polysaccharides, or biosaccharides, preferably, polysaccharides rich in rhamnose, galactose and glucuronic acid. A preferred polysaccharide is obtained by bacterial fermentation, and presents a branched structure and a sugar sequence rich in rhamnose (I, III, VI), galactose (II, V) and glucuronic acid (IV). Examples include biosaccharide gum-2, from less than 0.1%, sold under the tradename Rhamnosoft® HP, available from Solabia. When used as an ingredient in the skin care composition, it has been found that the biosaccharide gum-2 acts as a soothing and calming ingredient when applied to the skin and increases the overall performance of the skin care composition. Preferably, the polysaccharide is present in the composition from about 0.1% to about 4%, more preferably, from about 2% to about 3.5%, and most preferably, about 3%.

The skin care composition can further comprise additional ingredients to enhance the composition performance, stabilization, texture, viscosity and spreadability. Additional ingredients include, but are not limited to: moisturizing active ingredients, hydrators (e.g., dexpanthenol), penetration enhancers (e.g., tetrasodium glutamate), emulsifiers, thickeners, feel modifiers (e.g., silicones and elastomers), sensory enhancers (feel, texture, and smell enhancers), and stability enhancers. Other moisturizing active ingredients include occlusive, which provide a barrier on the skin that prevents trans epidermal water loss, lipids, which support the lipid lamellar layers between the corneocytes to increase barrier properties (e.g. ceramides, cholesterol and unsaturated fatty acids), and other natural moisturizing factors, including amino acids, lactates, sugars, urea, and inorganic salts, for example.

The composition is adjusted to a pH of between about 5 to about 6.5, preferably between about 6 to about 6.5. The composition has a final viscosity at 23 degrees Celsius, utilizing spindle 3 at 5 rpm, of between about 5000 to about 6500 and a specific gravity at 23 degrees Celsius of from about 1.00 to about 1.03. In a preferred embodiment, the skin care composition comprises sodium lactate, retinol, bakuchiol, azelaic acid, niacinamide, biosaccharide gum-2, and lactic acid. Other ingredients can include water, polyacrylate-13, cyclopentasiloxane, isoamyl laurate, magnesium aluminum silicate, panthenol, lecithin, glycerin, phenyl trimethicone, dicaprylyl carbonate, polyisobutene, dimethicone, polysorbate 80, hydroxyethylcellulose, polysorbate 20, pentylene glycol, phenoxyethanol, laureth-4, ethylhexylglycerin, tetrasodium glutamate diacetate, and potassium hydroxide. However, as will be understood by those of skill in the art with reference to this disclosure, other ingredients may be substituted and added to the composition formulation, as described herein.

This invention acts as an all in one solution by simplifying the skin care routine. The invention is a hybrid of 6 products, and the active ingredients treat rosacea, aging, texture, dehydration, dryness fine lines, wrinkles, skin tone, skin elasticity, psoriasis, skin cancer cells, age spots, sebaceous gland activity, acne, inflammation, eczema, dermatitis and overall skin health.

### Examples

Exemplary formulations of the skin care composition according to the invention are shown below in Table 1 and Table 2.

TABLE 1

| Formula | Wt % in | | Product | Ingredient |
|---|---|---|---|---|
| % | RM | Ingredient | Weight % | Range |
| to 100% | 100.0 | water | 75.5985 | >50% |
| 8.400 | 60.0 | sodium lactate | 5.0400 | 5-10% |
| 6.000 | 1.0 | retinol | 0.0600 | <0.1% |
| | 1.0 | polysorbate 20 | 0.0600 | <0.1% |
| | 1.0 | lecithin | 0.0600 | <0.1% |
| 4.000 | 100.0 | niacinamide | 4.0000 | 1.0-5% |
| 3.000 | 60.0 | polyacrylate-13 | 1.8000 | 1.0-5% |
| | 27.0 | polyisobutene | 0.8100 | 0.1-1% |
| | 5.0 | polysorbate 20 | 0.1500 | 0.1-1% |
| 3.000 | 2.5 | biosaccharide gum-2 | 0.0750 | <0.1% |
| 2.000 | 85.0 | cyclopentasiloxane | 1.7000 | 1.0-5% |
| | 15.0 | dimethicone | 0.3000 | 0.1-1% |
| 1.500 | 100.0 | magnesium aluminum | 1.5000 | 1.0-5% |
| | | silicate | | |
| 1.500 | 100.0 | isoamyl laurate | 1.5000 | 1.0-5% |
| 1.400 | 75.0 | panthenol | 1.0500 | 1.0-5% |
| 1.000 | 100.0 | glycerin | 1.0000 | 1.0-5% |
| 1.000 | 100.0 | pentylene glycol | 1.0000 | 1.0-5% |
| 0.900 | 100.0 | phenoxy ethanol | 0.9000 | 0.1-1% |
| 0.900 | 100.0 | phenyl trimethicone | 0.9000 | 0.1-1% |
| 0.600 | 100.0 | dicaprylyl carbonate | 0.6000 | 0.1-1% |
| 0.500 | 100.0 | bakuchiol | 0.5000 | 0.1-1% |
| 0.450 | 25.0 | potassium hydroxide | 0.1125 | 0.1-1% |
| 0.250 | 100.0 | polysorbate 80 | 0.2500 | 0.1-1% |
| 0.250 | 100.0 | hydroxyethylcellulose | 0.2500 | 0.1-1% |
| 0.200 | 100.0 | laureth-4 | 0.2000 | 0.1-1% |
| 0.200 | 100.0 | ethylhexylglycerin | 0.2000 | 0.1-1% |
| 0.200 | 47.0 | tetrasodium glutamate | 0.0940 | <0.1% |
| | | diacetate | | |
| 0.200 | 100.0 | azelaic acid | 0.2000 | 0.1-1% |
| 0.100 | 90.0 | lactic acid | 0.0900 | <0.1% |

The composition is adjusted to a pH of between 6 and 6.5

TABLE 2

| Formula | Wt % in | | Product | Ingredient |
|---|---|---|---|---|
| % | RM | Ingredient | Weight % | Range |
| to 100% | 100.0 | water | 75.5985 | >50% |
| 8.400 | 60.0 | sodium lactate | 5.0400 | 5-10% |
| 6.000 | 1.0 | tretinoin | 0.0600 | <0.1% |
| | 1.0 | polysorbate 20 | 0.0600 | <0.1% |
| | 1.0 | lecithin | 0.0600 | <0.1% |
| 4.000 | 100.0 | hydroquinone | 4.0000 | 1.0-5% |
| 3.000 | 60.0 | polyacrylate-13 | 1.8000 | 1.0-5% |
| | 27.0 | polyisobutene | 0.8100 | 0.1-1% |
| | 5.0 | polysorbate 20 | 0.1500 | 0.1-1% |
| 3.000 | 2.5 | biosaccharide gum-2 | 0.0750 | <0.1% |
| 2.000 | 85.0 | cyclopentasiloxane | 1.7000 | 1.0-5% |
| | 15.0 | dimethicone | 0.3000 | 0.1-1% |
| 1.500 | 100.0 | magnesium aluminum | 1.5000 | 1.0-5% |
| | | silicate | | |
| 1.500 | 100.0 | isoamyl laurate | 1.5000 | 1.0-5% |
| 1.400 | 75.0 | panthenol | 1.0500 | 1.0-5% |
| 1.000 | 100.0 | glycerin | 1.0000 | 1.0-5% |
| 1.000 | 100.0 | pentylene glycol | 1.0000 | 1.0-5% |
| 0.900 | 100.0 | phenoxyethanol | 0.9000 | 0.1-1% |
| 0.900 | 100.0 | phenyl trimethicone | 0.9000 | 0.1-1% |
| 0.600 | 100.0 | dicaprylyl carbonate | 0.6000 | 0.1-1% |
| 0.500 | 100.0 | bakuchiol | 0.5000 | 0.1-1% |
| 0.450 | 25.0 | potassium hydroxide | 0.1125 | 0.1-1% |
| 0.250 | 100.0 | polysorbate 80 | 0.2500 | 0.1-1% |
| 0.250 | 100.0 | hydroxyethylcellulose | 0.2500 | 0.1-1% |
| 0.200 | 100.0 | laureth-4 | 0.2000 | 0.1-1% |
| 0.200 | 100.0 | ethylhexylglycerin | 0.2000 | 0.1-1% |
| 0.200 | 47.0 | tetrasodium glutamate | 0.0940 | <0.1% |
| | | diacetate | | |
| 0.200 | 100.0 | azelaic acid | 0.2000 | 0.1-30% |
| 0.100 | 90.0 | lactic acid | 0.0900 | 0.05-30% |

The composition is adjusted to a pH of between 4 and 6.5

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A skin care composition comprising:
a) less than 0.05% lactic acid;
b) about 5% to about 10% sodium lactate;
c) about 1% to about 5% niacinamide;
d) less than about 0.1% biosaccharide gum-2;
e) about 0.1% to about 1% bakuchiol;
f) about 0.1% to about 1% azelaic acid;
g) less than about 0.1% retinol; and
h) less than about 0.1% lecithin.

2. The skin care composition of claim 1 comprising one or more additional ingredients selected from the group consisting of water, polyacrylate-13, cyclopentasiloxane, isoamyl laurate, magnesium aluminum silicate, panthenol, glycerin, phenyl trimethicone, dicaprylyl carbonate, polyisobutene, dimethicone, polysorbate 80, hydroxyethylcellulose, polysorbate 20, pentylene glycol, phenoxyethanol, laureth-4, ethylhexylglycerin, tetrasodium glutamate diacetate, and potassium hydroxide.

3. The skin care composition of claim 1, wherein the skin care composition further comprises more than about 50% water, about 1% to about 5% polyacrylate-13, about 0.1% to about 1% polyisobutene, about 0.1% to about 1% polysorbate 20, about 1% to about 5% cyclopentasiloxane, about 0.1% to about 1% dimethicone, about 1% to about 5% magnesium aluminum silicate, about 1% to about 5% isoamyl laurate, about 1% to about 5% panthenol, about 1% to about 5% glycerin, about 1% to about 5% pentylene glycol, about 0.1% to about 1% phenoxyethanol, about 0.1% to about 1% phenyl trimethicone, about 0.1% to about 1% dicaprylyl carbonate, about 0.1% to about 1% potassium hydroxide, about 0.1% to about 1% polysorbate 80, about 0.1%0 to about 1% hydroxyethylcellulose, about 0.1% to about 1% laureth-4, about 0.1% to about 1% ethylhexylglycerin, and less than about 0.1% tetrasodium glutamate diacetate.

4. A skin care composition comprising:
a) lactic acid;
b) sodium lactate;
c) niacinamide;
d) biosaccharide gum-2;
e) bakuchiol;
f) azelaic acid;
g) retinol; and
h) lecithin.

5. The skin care composition of claim 4, wherein the lactic acid comprises less than 0.1% of the skin care composition, the sodium lactate comprises about 5% to about 100% of the skin care composition, the niacinamide comprises from about 1% to about 5% of the skin care composition, the biosaccharide gum-2 comprises less than about 0.1% of the skin care composition, the bakuchiol comprises from about 0.1% to about 1% of the skin care composition, the azelaic acid comprises from about 0.1% to about 1% of the skin care composition, the retinol comprises less than 0.1% of the skin care composition, and the lecithin comprises less than about 0.1% of the skin care composition.

6. The skin care composition of claim 4 comprising one or more additional ingredients selected from the group consisting of water, polyacrylate-13, cyclopentasiloxane, isoamyl laurate, magnesium aluminum silicate, panthenol, glycerin, phenyl trimethicone, dicaprylyl carbonate, polyisobutene, dimethicone, polysorbate 80, hydroxyethylcellulose, polysorbate 20, pentylene glycol, phenoxyethanol, laureth-4, ethylhexylglycerin, tetrasodium glutamate diacetate, and potassium hydroxide.

7. The skin care composition of claim 4, wherein the skin care composition further comprises more than about 50% water, about 1% to about 5% polyacrylate-13, about 0.1% to about 1% polyisobutene, about 0.1% to about 1% polysorbate 20, about 1% to about 5% cyclopentasiloxane, about 0.1% to about 1% dimethicone, about 1% to about 5% magnesium aluminum silicate, about 1% to about 5% isoamyl laurate, about 1%/o to about 5% panthenol, about 1%/o to about 5% glycerin, about 1% to about 5% pentylene glycol, about 0.1% to about 1% phenoxyethanol, about 0.1% to about 1% phenyl trimethicone, about 0.1% to about 1% dicaprylyl carbonate, about 0.1% to about 1% potassium hydroxide, about 0.1% to about 1% polysorbate 80, about 0.1% to about 1% hydroxyethylcellulose, about 0.1% to about 1% laureth-4, about 0.1% to about 1% ethylhexylglycerin, and less than about 0.1% tetrasodium glutamate diacetate.

8. The skin care composition of claim 5, wherein the skin care composition further comprises more than about 50% water, about 1% to about 5% polyacrylate-13, about 0.1% to about 1% polyisobutene, about 0.1% to about 1% polysorbate 20, about 1% to about 5% cyclopentasiloxane, about 0.1% to about 1% dimethicone, about 1% to about 5% magnesium aluminum silicate, about 1% to about 5% isoamyl laurate, about 1% to about 5% panthenol, about 1% to about 5% glycerin, about 1% to about 5% pentylene glycol, about 0.1% to about 1% phenoxyethanol, about 0.1% to about 1% phenyl trimethicone, about 0.1% to about 1% dicaprylyl carbonate, about 0.1% to about 1% potassium hydroxide, about 0.1% to about 1% polysorbate 80, about 0.1% to about 1%/o hydroxyethylcellulose, about 0.1% to about 1% laureth-4, about 0.1% to about 1% ethylhexylglycerin, and less than about 0.1% tetrasodium glutamate diacetate.

9. A skin care composition comprising:
a) lactic acid;
b) niacinamide,
c) biosaccharide gum-2;
d) bakuchiol;
e) azelaic acid; and
f) retinol.

10. The skin care composition of claim 9, wherein the lactic acid comprises less than 0.1% of the skin care composition, the niacinamide comprises from about 1% to about 5% of the skin care composition, the biosaccharide gum-2 comprises less than about 0.1% of the skin care composition, the bakuchiol comprises from about 0.1% to about 1% of the skin care composition, the azelaic acid comprises from about 0.1% to about 1% of the skin care composition, and the retinol comprises less than 0.1% of the skin care composition.

11. The skin care composition of claim 9 comprising one or more additional ingredients selected from the group consisting of water, polyacrylate-13, cyclopentasiloxane, isoamyl laurate, magnesium aluminum silicate, panthenol, glycerin, phenyl trimethicone, dicaprylyl carbonate, polyisobutene, dimethicone, polysorbate 80, hydroxyethylcellulose, polysorbate 20, pentylene glycol, phenoxyethanol, laureth-4, ethylhexylglycerin, tetrasodium glutamate diacetate, potassium hydroxide, sodium lactate and lecithin.

12. The skin care composition of claim 4, wherein the skin care composition further comprises more than about 50% water, about 1% to about 5% polyacrylate-13, about 0.1% to about 1% polyisobutene, about 0.1% to about 1% polysorbate 20, about 1% to about 5% cyclopentasiloxane, about 0.1% to about 1% dimethicone, about 1% to about 5% magnesium aluminum silicate, about 1% to about 5% isoamyl laurate, about 1% to about 5% panthenol, about 1% to about 5% glycerin, about 1% to about 5% pentylene glycol, about 0.1% to about 1% phenoxyethanol, about 0.1% to about 1% phenyl trimethicone, about 0.1% to about 1% dicaprylyl carbonate, about 0.1% to about 1% potassium hydroxide, about 0.1% to about 1% polysorbate 80, about 0.1% to about 1% hydroxyethylcellulose, about 0.1% to about 1° % laureth-4, about 0.1% to about 1% ethylhexylglycerin, and less than about 0.1% tetrasodium glutamate diacetate, about 5% to about 10% sodium lactate and less than about 0.1% lecithin.
